(19) United States
(12) Patent Application Publication
(10) Pub. No.: US 2026/0020814 A1
(43) Pub. Date: Jan. 22, 2026

(54) Smart Oral Health Monitoring System and Method Using Miniature Wireless Sensors

(71) Applicant: OHANNES OHANNESSIAN, Round Rock, TX (US)

(72) Inventor: OHANNES OHANNESSIAN, Round Rock, TX (US)

(21) Appl. No.: 18/774,759

(22) Filed: Jul. 16, 2024

(51) Int. Cl. A61B 5/00

(52) U.S. Cl. CPC A61B 5/4547; A61B 5/0002; A61B 5/4519; A61B 5/682; A61B 2562/06; A61B 2562/164

(57) ABSTRACT

The present invention relates to a miniature wireless teeth sensor system and method for comprehensive, real-time monitoring of oral health parameters. The system comprises a plurality of smart biocompatible sensor units configured for placement on or integration into teeth or dental appliances, wireless transmission modules, and a receiver. The sensor units incorporate one or more sensors adapted to detect and monitor various oral health parameters. The invention encompasses active capsules with built-in batteries, distributed multi-sensor arrays, and standalone, compact, all-in-one sensor units. Data transmission is achieved through wireless communication technologies. The invention also provides a method for monitoring oral health using the described system. This invention represents a significant advancement in dental healthcare technology, enabling early detection of oral health issues and personalized treatment planning.

### BACKGROUND

### Field of the Invention

The present invention relates to the field of dental healthcare technology, and more specifically, to miniature wireless teeth sensors for continuous monitoring of oral health parameters to support teeth alignment and adjustment.

### Description of Related Art

In recent years, there has been a growing emphasis on leveraging technology to enhance preventive and personalized healthcare solutions. Traditional methods of monitoring oral health often lack real-time data and require periodic, sometimes invasive, check-ups. This limitation has led to the development of various intraoral monitoring devices and systems aimed at providing continuous, non-intrusive monitoring of oral health parameters.

One such example is disclosed in U.S. Patent Application Publication No. 2023/0190195, which describes an intraoral monitoring device for a mouthpiece adapted to be received intraorally in a dentition of a user. The device comprises a flexible substrate coupled to the mouthpiece and a plurality of sensors integrated on a flexible printed circuit board (PCB). The sensors include a first photoplethysmography (PPG) sensor located along the mid-line of the maxilla to provide signals related to cardiorespiratory parameters, and a second PPG sensor located within the oral cavity to provide signals related to muscular parameters. While this device enables monitoring of various physiological parameters during sleep, it does not specifically address the need for continuous monitoring of oral health parameters to support teeth alignment and adjustment.

Miniature wireless teeth sensors represent a convergence of various technical domains, including microelectronics, sensor technology, wireless communication, biosensors, and materials science. These sensors are typically equipped with miniaturized components such as micro-sensors to measure parameters like pH levels, temperature, and friction pressure between upper and bottom teeth within the oral cavity. Biosensors enable simple and non-invasive collection methods, allowing for easy and fast diagnostic testing of the oral environment, which contains salivary secretions, blood supply, lymph nodes, ingested pathogens, toxins, allergens, drugs, nutrients, food constituents, humidity, and acidity. The presence of various biomarkers permits accurate reflection of normal and disease states.

The primary problem addressed by miniature wireless teeth sensors is the need for continuous and real-time monitoring of oral health to support teeth alignment and adjustment. Traditional dental check-ups are periodic and might miss crucial changes in oral conditions between visits. Miniature wireless teeth sensors provide a solution by offering a constant stream of data, enabling dentists to monitor patients' oral health remotely. This continuous monitoring is particularly crucial for early detection of issues such as teeth friction, misalignment, tooth decay, gum diseases, or changes in oral pH levels, allowing for timely interventions and personalized treatment plans. Additionally, these sensors empower individuals to actively engage in their oral healthcare by providing them with data-driven insights, fostering preventive dental practices and encouraging healthier lifestyles.

In summary, miniature wireless teeth sensors represent a cutting-edge advancement in dental healthcare, integrating sensor technology and wireless communication to provide real-time data for both dentists and patients. By addressing the limitations of traditional monitoring methods and existing intraoral monitoring devices, these sensors facilitate early detection, personalized treatment, and preventive dentistry, ultimately improving oral health outcomes and patient well-being, particularly in the context of supporting teeth alignment and adjustment.

### SUMMARY

The present invention provides a smart miniature wireless teeth sensor system and method for comprehensive, real-time monitoring of oral health parameters. The system comprises a plurality of biocompatible smart sensor units, wireless transmission modules, and a receiver, enabling continuous data collection and analysis to facilitate dental diagnosis, treatment, and monitoring.

In one aspect, the invention includes smart biocompatible sensor units configured for placement on or integration into teeth or dental appliances within an oral cavity. These smart sensor units incorporate one or more sensors adapted to detect and monitor various oral health parameters, including but not limited to temperature, pressure, pH level, and bacterial presence. The system's versatility is further enhanced by additional smart sensors capable of detecting physiological parameters such as blood pressure, heart rate, and respiration rate.

A key feature of the invention is its adaptability to various dental contexts. The smart biocompatible sensor units can be integrated into a range of dental appliances, including retainers, braces, crowns, and bridges. Alternatively, they may be designed as ultra-thin, flexible smart bio-tags that conform to tooth surfaces, suitable for temporary monitoring or extended wear. The invention also encompasses smart miniature sensor capsules encased in dental composites, which can be integrated during dental treatments such as fillings and bondings.

The system's smart architecture is flexible, allowing for various configurations to suit different monitoring needs. These configurations include smart active capsules with built-in batteries, distributed smart multi-sensor arrays connected via thin wires to a central processing unit, and standalone, compact, all-in-one smart sensor units that can be placed according to specific requirements.

Data transmission is achieved through smart wireless communication technologies such as Bluetooth, RFID, NFC, or WiFi, enabling seamless connectivity with receivers such as smartphones or dedicated smart monitoring devices. This feature allows for real-time monitoring and analysis of oral health parameters by users or dental professionals.

The invention also encompasses a smart method for monitoring oral health using the described system. This method involves placing or integrating the smart sensor units, operating them to collect data, wirelessly transmitting the data, analyzing it to monitor oral health status, and utilizing the analyzed data to inform dental diagnosis and treatment planning.

Advanced smart features of the invention include the potential for energy harvesting from oral cavity movements or temperature differentials, the use of machine learning algorithms for pattern identification and prediction of dental issues, and the capability to generate smart alerts when predetermined oral health parameter thresholds are exceeded.

By providing continuous, real-time smart monitoring of multiple oral health parameters, this invention represents a significant advancement in dental healthcare technology. It offers the potential for early detection of oral health issues, personalized treatment planning, enhanced patient experience, and improved patient engagement in oral healthcare, ultimately leading to better dental outcomes and overall well-being.

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 illustrates a block diagram of an embodiment of a miniature wireless teeth sensor system.

FIG. 2 illustrates an embodiment of a flow diagram detailing the important steps and processes involved in the operation of the miniature wireless teeth sensor system. longevity of the monitoring system.

FIG. 3 illustrates an embodiment of a detailed view of the placement and configuration of the miniature wireless teeth sensor system within a patient's oral cavity.

FIG. 4 illustrates an embodiment of various placement options for the smart biocompatible sensor units of the miniature wireless teeth sensor system.

FIG. 5 illustrates an embodiment of a comprehensive electronic system architecture for the miniature wireless teeth sensor system.

### DETAILED DESCRIPTION

In the following detailed description of the embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word or as used herein means any one member of a particular list and also includes any combination of members of that list. Further, one should note that conditional language, such as, among others, “can,” “could,” “might”, or “may” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

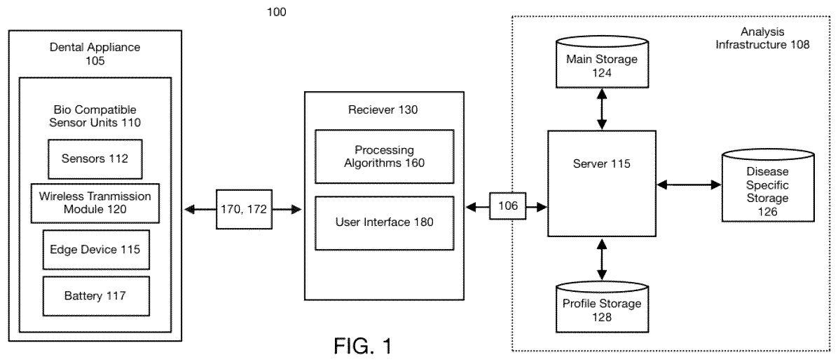

FIG. 1 illustrates a block diagram of an embodiment of a miniature wireless teeth sensor system 100. The system 100 comprises a plurality of smart biocompatible sensor units 110 configured for placement on or integration into one or more teeth or dental appliances 105 within an oral cavity. Each sensor unit 110 includes one or more sensors 112 adapted to detect and monitor various oral health parameters such as temperature, pressure, pH level, bacterial presence, blood pressure, heart rate, respiration rate, and levels of predetermined biologics, chemicals, or medications.

The sensors 112 can be microelectromechanical systems (MEMS) based sensors, such as piezoresistive pressure sensors, thermistors or thermocouples for temperature sensing, ion-sensitive field-effect transistors (ISFETs) for pH measurement, and electrochemical sensors for detecting specific biomarkers or bacteria. These miniaturized sensors enable non-invasive, continuous monitoring of the oral environment. The sensors can also include accelerometers, gyroscopes, magnetometers, and other types of sensors to measure physical properties and motion within the oral cavity.

Each sensor unit 110 is associated with a wireless transmission module 120 that transmits the data collected by the sensors 112 to a receiver 130. The wireless transmission module 120 can utilize various wireless communication technologies such as Bluetooth, radio-frequency identification (RFID), near-field communication (NFC), or WiFi, depending on power consumption and data transmission requirements. For example, Bluetooth Low Energy (BLE) offers low power consumption suitable for battery-operated sensor units, while NFC allows battery-free operation by harvesting energy from an external reader device.

The sensor units 110 can also include an micro controller component 115 that runs lightweight machine learning algorithms to perform local data processing and decision making. This allows the sensor units to intelligently determine when to transmit data based on predefined thresholds or detected anomalies, optimizing power consumption and data transmission efficiency.

The transmitted sensor data is received by the receiver 130, which can be a smartphone running a dedicated application or a custom monitoring device. The receiver 130 processes and analyzes the received data to provide real-time information about the monitored oral health parameters. This enables users or dental professionals to track changes, detect anomalies, and make informed decisions regarding dental diagnosis, treatment, and monitoring.

The system 100 can be implemented using various form factors for the sensor units 110. One approach is to embed the sensors and wireless transmission module into dental appliances 105 like braces, retainers, or mouthguards. This allows seamless integration without affecting the user's comfort or appearance. In this embodiment the power source is implemented using a mini lithium ion battery 117.

The choice of power source for the sensor units depends on the specific application and desired operating lifetime. Active capsules 140 with batteries offer continuous monitoring but may require periodic replacement. Passive capsules 150 eliminate the need for battery replacement but rely on proximity to an external reader for power and data transfer. Hybrid approaches can also be employed, such as using a rechargeable battery that is wirelessly charged through NFC or RFID.

In some embodiments the system supports various data streaming modes 172 to accommodate different use cases and connectivity scenarios. Real-time streaming enables immediate data transmission and analysis for time-critical applications. Near-time streaming allows for slightly delayed but prompt processing when real-time transmission is not essential. Offline streaming stores data for later analysis when connectivity is unavailable, ensuring data capture even in remote or disconnected environments. The data streaming modes 172 are managed by the receiver 130 and the server 115, which coordinate the transmission and storage of data based on the available connectivity and user preferences.

The data storage and analysis infrastructure 108 includes a main storage 124 that serves as the primary data repository. The main storage 124 employs encryption techniques to ensure data security and privacy. The system also incorporates a disease group specific storage database 126 which stores disease-related information for targeted analysis and research. Additionally, an Individual specific profile storage 128 maintains personalized data, including sensor profile IDs, historical records, performance statistics, and individual/group IDs. The server 116 manages the data storage and analysis infrastructure 108, ensuring efficient data organization, retrieval, and processing.

The analysis Infrastructure 108 of the system can utilize the sensor data for various functions, including imaging, data storage and retrieval, alert generation, data management, processing, modeling, statistical analysis, and data visualization. The system can also activate specific sensors based on triggers from other sensors or remote commands.

The system's output and applications encompass a wide range of functionalities. It provides advanced analytics for in-depth analysis of the collected oral health data. Historical data is stored and trended over time to identify long-term patterns and changes in the user's oral health metrics. The system enables continuous tracking of specific oral health parameters for monitoring purposes. It generates alerts for significant events or concerning trends detected in the user's oral health data. These alerts are communicated to the user via the receiver 130 and can also be shared with healthcare providers for timely intervention.

The wearable dental system supports preventive dental care by facilitating early detection of oral health issues through continuous monitoring and analysis. It also enables personalized treatment planning by leveraging individual data to tailor interventions and optimize dental care outcomes. The server (116) processes the data using advanced algorithms and generates personalized recommendations and treatment plans based on the user's specific oral health profile.

As mentioned above the smart biocompatible sensor units 110 can be integrated into various dental appliances 105 to enable continuous monitoring of oral health parameters. In one embodiment, the sensor units are embedded into dental appliances 105 selected from the group consisting of retainers, braces, crowns, and bridges. For example, the sensors 112 and wireless transmission module 120 can be incorporated into the structure of a retainer during the manufacturing process. This allows the retainer to serve its primary function of maintaining tooth alignment while simultaneously monitoring the oral environment. Similarly, the sensor units can be integrated into the brackets or wires of dental braces, the material of dental crowns, or the framework of dental bridges. By integrating the sensor units into existing dental appliances 105, the system 100 enables seamless and non-invasive monitoring without requiring additional devices to be worn.

In another embodiment (not shown), the smart biocompatible sensor units 110 comprise ultra-thin, flexible bio-tags that conform to tooth surfaces and are suitable for temporary monitoring or extended wear. These bio-tags can be made from soft, stretchable materials such as silicone elastomers or thermoplastic polyurethanes that can adapt to the contours of individual teeth. The sensors 112 and wireless transmission module 120 are embedded within the flexible substrate of the bio-tags, allowing them to maintain contact with the tooth surface. The bio-tags can be applied to the teeth using a mild adhesive and can be easily removed by the user or a dental professional when monitoring is complete. The ultra-thin and flexible nature of the bio-tags ensures comfort during wear and minimizes interference with normal oral functions such as speaking and eating.

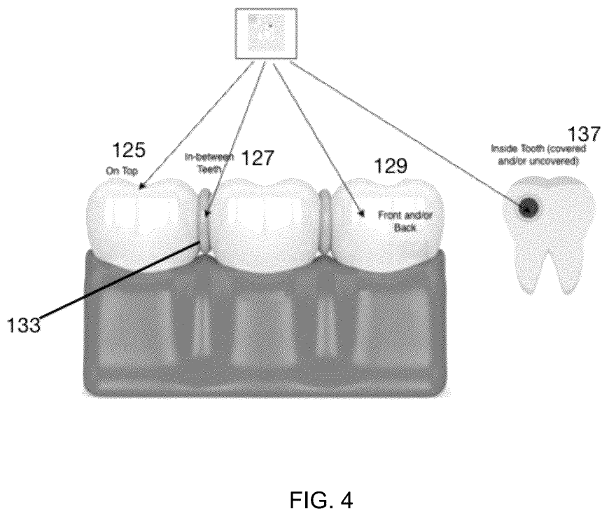

In yet another embodiment, the smart biocompatible sensor units 110 comprise miniature sensor capsules encased in dental composites and integrated during dental treatments 137 such as fillings and bandings as depicted in FIG. 4. The sensor capsules can be made from biocompatible materials like glass or ceramic and contain the sensors 112 and wireless transmission module 120. During a dental filling or bonding procedure, the dentist can place the sensor capsule into the prepared cavity or onto the tooth surface before applying the dental composite material. The composite material, such as resin or glass ionomer cement, encapsulates the sensor capsule and secures it in place. This approach allows the integration of monitoring capabilities during routine dental treatments, enabling long-term tracking of oral health parameters at specific tooth locations.

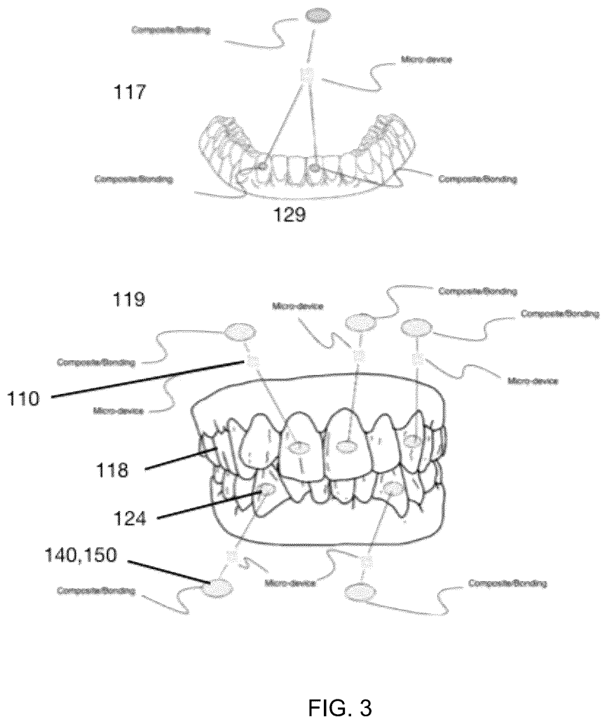

In some embodiments the sensor units 110 can be miniature sensor capsules as depicted in FIG. 3 that are temporarily attached to the teeth 118 using dental adhesives 124. These capsules can be designed as active capsules 140 with built-in batteries for continuous operation, transmitting data via Bluetooth or RFID to the receiver. Passive capsules 150 without batteries can also be used, relying on NFC or RFID for power and data transmission when interrogated by an external reader.

In one embodiment (now shown) the smart biocompatible sensor units 110 can also be configured as a distributed multi-sensor array including a network of sensors placed across multiple teeth and connected via thin wires to a central processing unit. In this embodiment, individual sensor units are placed on or attached to different teeth, with each sensor unit containing one or more sensors 112 specialized for detecting specific oral health parameters. The sensor units are interconnected using thin, flexible wires that run along the gum line or are embedded within dental appliances. The wires transmit the sensor data to a microcontroller 115, which can be located within a dental appliance or externally in the oral cavity. The central processing unit aggregates the data from all the sensor units, performs any necessary processing or analysis, and transmits the information wirelessly to the receiver 130 using the wireless transmission module 120. This distributed multi-sensor array approach enables comprehensive monitoring of the entire oral cavity and can provide a more detailed spatial map of oral health parameters.

In another embodiment, the smart biocompatible sensor units 110 comprise standalone, compact, all-in-one sensor units 133 placed according to need. These standalone sensor units 133 are designed to attach directly to tooth surfaces or fit between teeth and operate independently without requiring connections to other components as depicted in FIG. 3. Each standalone sensor unit 133 includes the necessary sensors 112, wireless transmission module 120, and an on-board battery 117 within a small, self-contained package. The sensor units 110 can be attached to the teeth using dental adhesives or mechanically secured in place by their shape and size. For example, a standalone sensor unit can be shaped like a small cylinder or disk that fits snugly between two teeth 127, held in place by the natural contours of the teeth. The on-board battery 117 provides power for the sensors and wireless transmission, allowing the standalone sensor units to operate continuously for a predetermined period before requiring replacement. The wireless transmitter 120 sends the collected data directly to the receiver 130, eliminating the need for any wired connections. This embodiment offers flexibility in sensor placement and enables targeted monitoring of specific areas of interest within the oral cavity.

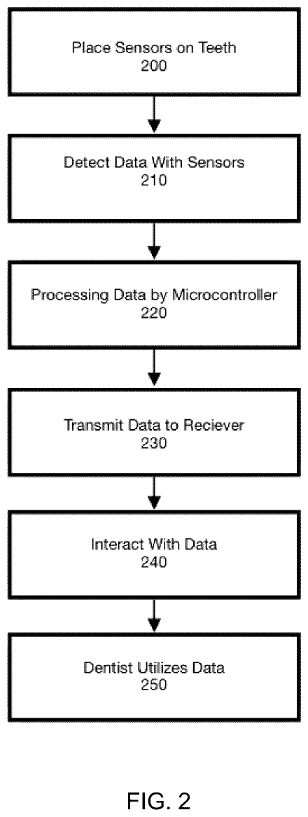

FIG. 2 illustrates a flow diagram detailing the important steps and processes involved in the operation of the miniature wireless teeth sensor system. The system comprises a plurality of biocompatible miniature wireless sensor units 110 placed or integrated on or into one or more teeth 118 or dental appliances within the oral cavity of a patient. Each sensor unit 110 includes one or more sensors 112 configured to detect and monitor various oral health parameters.

During data collection 210, the sensors 112 continuously or intermittently collect data on the oral health parameters of interest. These sensors 112 may include, but are not limited to, pH sensors, temperature sensors, pressure sensors, accelerometers, gyroscopes, and chemical sensors capable of detecting specific biomarkers in saliva. The sensors 112 are designed to be highly sensitive and accurate, enabling the capture of precise data on the oral environment.

Once the raw data is collected, it is processed by a microcontroller 115 within each sensor unit 110 for data processing 220. The microcontroller 115 applies light weight machine learning algorithms to generate processed data from the collected sensor data. This processed data can be used to determine meaningful information about the user's oral health status. This processing step may involve signal conditioning, data filtering, and the application of machine learning techniques to identify patterns and anomalies in the data.

In some embodiments the processed data generated by the microcontroller 115 can be utilized to enable auto-adjustment of retainers, braces, or other dental appliances 105, or to request manual adjustments based on predefined settings and requirements. The system can compare the processed data against preset thresholds or patterns that indicate the need for adjustments. For instance, if the processed data suggests a significant deviation from the expected tooth alignment trajectory, the microcontroller 115 can send a signal to the dental appliance 105 to initiate an automatic adjustment. This auto-adjustment mechanism may involve the use of shape memory alloys, micro-actuators, or other smart materials embedded within the dental appliance 105.

Alternatively, if the deviation exceeds a certain level or if auto-adjustment is not feasible, the system can generate an alert or notification for the user and/or the dental healthcare provider, prompting a manual adjustment of the dental appliance 105. The predefined settings and requirements that trigger these adjustments can be customized based on the user's specific treatment plan, dental history, and progress.

After processing, the data is ready for wireless transmission 230 to a receiver, such as a smartphone or a web portal. Each sensor unit 110 is equipped with a wireless transmission module 120 that utilizes wireless communication protocols 170 like Bluetooth, RFID, NFC, or WiFi to transmit the processed data securely and efficiently. The wireless transmission module 120 is designed to consume minimal power, ensuring long battery life for the sensor units 110.

Upon receiving the transmitted data, users and healthcare providers can interact with the miniature wireless teeth sensor system 240 through a user-friendly interface 180. The interface may be in the form of a smartphone app or a web portal, providing intuitive visualizations of the real-time data and historical trends. Users can easily interpret the data and gain valuable insights into their oral health status. The interface may also include features for setting personalized alerts and notifications when certain oral health parameters exceed predetermined thresholds.

The analyzed data generated by the miniature wireless teeth sensor system serves as a powerful tool for dental professionals. Dentists can utilize this data to inform their diagnoses, develop personalized treatment plans, and provide early intervention for dental issues 250. By continuously monitoring key oral health parameters, the system enables the detection of subtle changes or abnormalities that may otherwise go unnoticed, facilitating proactive dental care.

To ensure uninterrupted operation, some sensor units 110 may be powered by energy harvesting mechanisms that convert oral cavity movements or temperature differentials into electrical energy. This eliminates the need for frequent battery replacements and enhances the convenience and longevity of the monitoring system.

FIG. 3 illustrates a detailed view of the placement and configuration of the miniature wireless teeth sensor system 100 within a patient's oral cavity. The upper portion of the figure depicts a single dental arch, representing the lower jaw 117, while the lower portion shows a complete set of upper and lower teeth.

The sensor units 110 are strategically positioned at various points on the dental structures. In the upper portion 117, two sensor units 110 are visible, placed on the front teeth 129. The lower view 119 displays multiple sensor units 110 distributed across both the upper and lower teeth, including incisors, canines, and molars in both arches.

Each sensor unit 110 is securely attached to the teeth using a removable composite bonding. The sensor units 110 are equipped with advanced sensing capabilities, enabling them to monitor various oral health parameters in real-time. These parameters may include tooth movement, pressure distribution, wear time of the retainer, oral hygiene conditions, temperature, and pH levels within the oral cavity.

The strategic placement of the sensor units 110 across different teeth ensures comprehensive coverage and accurate data collection. The sensors 112 positioned on the front teeth may focus on monitoring tooth movement and alignment, while those placed on the canines and molars may prioritize factors such as pressure distribution and wear time.

FIG. 4 illustrates various placement options for the smart biocompatible sensor units 110 of the miniature wireless teeth sensor system 100. The sensor units 110 are strategically positioned to enable comprehensive monitoring of oral health parameters, as described herein.

One potential placement for the sensor units 110 is on the top surface of a tooth 125. This placement allows the sensors 112 within the sensor unit 110 to detect and monitor parameters such as temperature, pressure, pH level, and bacterial presence at the occlusal surface of the tooth.

The sensor units 110 can also be placed in the interdental spaces between teeth 127, as depicted by the small blue devices in the main illustration. This placement enables the sensors 112 to monitor oral health parameters within the interproximal regions, providing valuable data on the conditions between teeth.

Additionally, the sensor units 110 can be positioned on the front (labial or buccal) and/or back (lingual or palatal) surfaces of teeth, as indicated in the diagram 129. These placements allow for monitoring of oral health parameters at different locations along the tooth surfaces, enabling a more comprehensive assessment of dental health.

The bottom-right illustration shows a cross-section of a tooth with a dark spot inside, indicating that the sensor units 110 can potentially be embedded within the tooth structure itself. This placement option allows for monitoring of internal tooth conditions, such as temperature and pressure changes, which may be relevant for certain dental diagnoses or treatments.

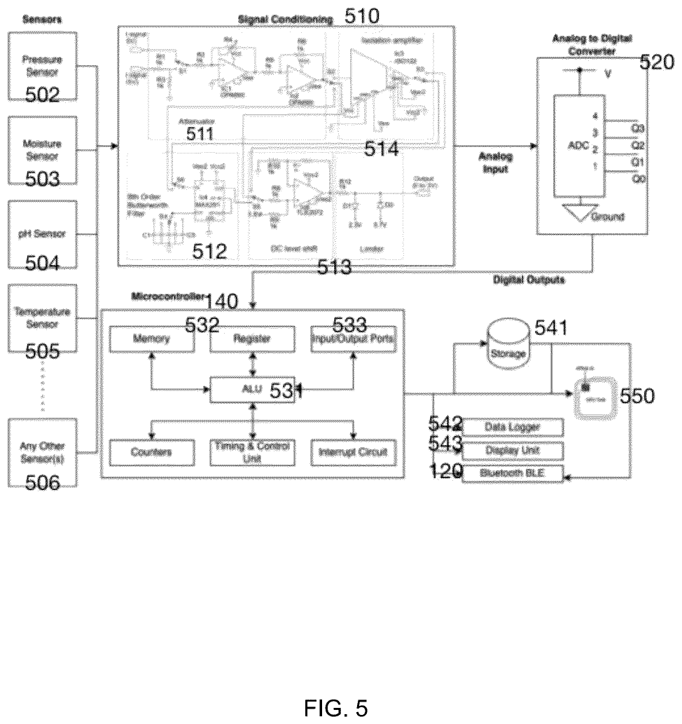

FIG. 5 illustrates a comprehensive embodiment of a electronic system architecture for the miniature wireless teeth sensor system 100. The circuit design enables the collection, processing, and transmission of various oral health parameters in real-time, supporting the monitoring of teeth alignment during retainer use.

The system includes multiple sensors 112 for monitoring various oral health parameters. A pressure sensor 502 measures teeth pressures during alignment, a moisture sensor 503 assesses oral hygiene or saliva production, a pH sensor 504 monitors oral pH levels, and a temperature sensor 505 corresponds to the temperature monitoring capability. The system also allows for flexibility to include any other sensors 506, possibly for detecting bacterial presence.

The signal conditioning section 510 processes the raw sensor signals. It includes an attenuator 511 to adjust signal strength, an 8th order Butterworth filter 512 for signal filtering, a DC level shift and limiter 513 to ensure the signal is within the appropriate range for further processing, and an isolation amplifier 514 to protect the circuit from electrical interference.

An analog-to-digital converter (ADC) 520 converts the processed analog signals from the sensors into digital data that can be interpreted by the nano/microcontroller 140. The microcontroller 115 contains an arithmetic logic unit (ALU) 531 for performing calculations on the sensor data, memory and registers 532 for storing data and instructions, input/output ports 533 for interfacing with sensors and output devices, and counters, timing & control unit, and interrupt circuit 534 for managing the system's operations and timing.

The digital outputs are part of the data processing algorithms 160 which include storage 541 for temporary data storage before transmission, a data logger 542 for recording sensor 112 readings over time, a display unit 543 for immediate data visualization, and a wireless transmission module 120 with Bluetooth BLE capability for enabling wireless transmission of data to a receiver 130.

Additional features include an NFC tag (550) for near-field communication capability, possibly for easy pairing or data transfer to dental equipment.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A miniature wireless teeth sensor system comprising:
a plurality of smart biocompatible sensor units configured for placement on or integration into one or more teeth or dental appliances within an oral cavity, each sensor unit comprising one or more sensors adapted to detect and monitor one or more oral health parameters comprising temperature, pressure, pH level, and bacterial presence;
a microcontroller embedded with lightweight machine learning algorithms to analyze the sensor data and apply behavioral actions based on predefined settings and requirements;
a wireless transmission module associated with each sensor unit, the wireless transmission module configured to transmit data collected by the one or more sensors; and
a receiver configured to receive the transmitted data from the wireless transmission modules;
wherein the transmitted data provides real-time information regarding the one or more monitored oral health parameters to facilitate dental diagnosis, treatment, and monitoring.

2. The system of claim 1, wherein the one or more sensors further comprise sensors for detecting blood pressure, heart rate, respiration rate, and levels of predetermined biologics, chemicals, or medications.

3. The system of claim 1, wherein the smart biocompatible sensor units are integrated into dental appliances selected from the group consisting of retainers, braces, crowns, and bridges.

4. The system of claim 1, wherein the smart biocompatible sensor units comprise ultra-thin, flexible bio-tags that conform to tooth surfaces and are suitable for temporary monitoring or extended wear.

5. The system of claim 1, wherein the smart biocompatible sensor units comprise miniature sensor capsules encased in dental composites and integrated during dental treatments selected from the group consisting of fillings and bondings.

6. The system of claim 5, wherein the miniature sensor capsules comprise active capsules with built-in batteries for continuous operation and transmit data via Bluetooth or RFID to the receiver.

7. The system of claim 1, wherein the smart biocompatible sensor units comprise a distributed multi-sensor array including a network of sensors placed across multiple teeth and connected via thin wires to a central processing unit.

8. The system of claim 1, wherein the smart biocompatible sensor units comprise standalone, compact, all-in-one sensor units placed according to need, wherein the standalone sensor units attach to tooth surfaces or fit between teeth and include an on-board battery and wireless transmitter.

9. The system of claim 1, wherein the wireless transmission module utilizes a wireless communication technology selected from the group consisting of Bluetooth, RFID, NFC, and WiFi.

10. The system of claim 1, wherein the receiver is a smartphone or a dedicated monitoring device that receives the transmitted data, enabling real-time monitoring and analysis of the one or more oral health parameters by a user or dental professional.

11. A method for monitoring oral health, the method comprising:
placing or integrating a plurality of biocompatible miniature wireless sensor units on or into one or more teeth or dental appliances within an oral cavity of a patient, each sensor unit comprising one or more sensors configured to detect and monitor one or more oral health parameters;
operating the plurality of sensor units to continuously or intermittently collect data on the one or more oral health parameters;
generating, by a microcontroller embedded with lightweight machine learning algorithms using the collected data, processed data;
applying behavioral actions based on predefined settings, requirements and the processed data;
wirelessly transmitting the collected and processed data from the sensor units to a receiver using wireless transmission modules associated with each sensor unit;
analyzing the transmitted data to monitor the patient's oral health status and detect changes or abnormalities; and
utilizing the analyzed data to inform dental diagnosis, generate personalized treatment plans, and provide early intervention for dental issues;
thereby facilitating comprehensive, real-time monitoring of the patient's oral health for improved dental outcomes.

12. The method of claim 11, wherein the one or more sensors comprise at least one of: a pH sensor, a temperature sensor, a pressure sensor, an accelerometer, a gyroscope, or a chemical sensor configured to detect specific biomarkers in saliva.

13. The method of claim 11, wherein at least one of the plurality of sensor units is integrated into a dental appliance selected from the group consisting of: a retainer, a brace, a crown, a bridge, and an aligner.

14. The method of claim 11, wherein at least one of the plurality of sensor units comprises an ultra-thin, flexible bio-tag conformally adhered to a tooth surface.

15. The method of claim 11, wherein at least one of the plurality of sensor units comprises an embedded sensor capsule integrated within a dental composite material.

16. The method of claim 11, wherein the wireless transmission modules utilize at least one of: Bluetooth, RFID, NFC, or WiFi technology to transmit the collected data.

17. The method of claim 11, further comprising powering at least one of the plurality of biocompatible miniature wireless sensor units using energy harvesting from oral cavity movements or temperature differentials.

18. The method of claim 11, wherein analyzing the transmitted data comprises utilizing machine learning algorithms to identify patterns indicative of oral health issues or to predict future dental problems.

19. The method of claim 11, further comprising generating alerts or notifications based on the analyzed data when predetermined oral health parameter thresholds are exceeded.

20. The method of claim 11, wherein at least one of the plurality of biocompatible miniature wireless sensor units is configured to detect and monitor muscle tone or weakness in the oral cavity, thereby providing data relevant to orthodontic treatment progress or temporomandibular joint disorders.
